(19) United States
(12) Patent Application Publication
(10) Pub. No.: US 2011/0059188 A1
(43) Pub. Date: Mar. 10, 2011

(54) COMPOSITIONS FOR REDUCING DENTAL EROSION

(76) Inventors: Michele Emily Barbour, Avon (GB); Robert Peter Shellis, Avon (GB)

(21) Appl. No.: 12/744,887

(22) PCT Filed: Nov. 27, 2008
(86) PCT No.: PCT/EP08/66310

§ 371 Date: Nov. 22, 2010

(30) Foreign Application Priority Data
Nov. 30, 2007 (GB) 0723519.5

(51) Int. Cl. A61K 33/06; A61K 38/00

(52) U.S. Cl. 424/682; 514/16.7

(57) ABSTRACT

The present invention relates to the use of a protein selected from one or more of casein, ovalbumin, whey protein and soy protein for the reduction of dental erosion caused by exposure to acidic compositions having a pH in the range 2.0 to 5.0.

The present invention relates to the use of a protein selected from one or more of casein, ovalbumin, whey protein and soy protein for the reduction of dental erosion caused by exposure to acidic compositions having a pH in the range 2.0 to 5.0.

Dental erosion describes the “pathologic, chronic, localised, painless loss of dental hard tissue chemically etched away from the tooth surface by acid and/or chelation without bacterial involvement” (Imfeld, 1996, Eur J. Oral Sci. 104, 151-155.). The acids causing the erosion are derived from dietary, occupational or intrinsic sources and are not products of the intraoral flora. Therefore dental erosion is a condition distinct from and different to dental caries with dis-similar etiology. It is thought that erosion of teeth is caused inter alia by acidic foodstuffs leaching out calcium from the teeth faster than it can be replaced by normal remineralisation processes. When an acidic product such as a beverage is prepared in accordance with this invention, and introduced into the oral cavity for consumption or healthcare purposes, the dissolution or removal of calcium and phosphate from teeth by chemical processes is significantly reduced.

Lussi et al (1995, Caries Res 29, 349-354) have associated the pH and titratable acidity of a beverage with its erosive potential; the greater the concentration of acid in the beverage the more damaging to teeth it became. Similarly a study in children (Millward et al, (1994) Int. J. Paed. Dent. 4, 151-157.) associated the presence of dental erosion with the consumption of acidic beverages and fruit juices.

When a product such as a beverage is prepared in accordance with this invention, and introduced into the oral cavity for consumption or healthcare purposes, the dissolution or removal of calcium and phosphate from teeth by chemical processes is significantly reduced.

It has been claimed that an increased intake of dietary acids, and a move away from formalised meal times, has been accompanied by a rise in the incidence of dental erosion. In view of this, methods which help prevent dental erosion and tooth wear would be advantageous.

EP 1 625 877A, (Friesland Brands BV), describes a protein-free, milk whey fraction, which can be used to reduce the negative effects associated with the oral uptake of acidic foods and beverages.

The present invention is based on the discovery that certain proteins have the ability to reduce or alleviate dental erosion caused by the intake of dietary acids.

In a first aspect, the present invention provides the use of a protein selected from one or more of casein, ovalbumin, whey protein and soy protein in the manufacture of an orally administrable composition comprising an acidulant and having a pH in the range 2.0 to 5.0, for the reduction of dental erosion caused by exposure to the acidulant.

In another aspect, there is provided an orally administrable composition comprising an acidulant and a protein selected from one or more of casein, ovalbumin, whey protein and soy protein and having a pH in the range 2.0 to 5.0 for use in the reduction of dental erosion caused by exposure to the acidulant.

The protein for use in acidic compositions according to the invention is present in an amount from 0.01% w/v to 50.0% w/v, suitably 0.01% w/v to 1.0 w/v, 0.05% w/v to 0.5% w/v, 0.1% w/v to 25.0% w/v and 2.0% w/v to 10.0% w/v.

Typically the amount of protein present in compositions for use in the invention will vary from 0.10 g/L to 500 g/L, suitably 0.1 g/L to 10 g/L, 0.5 g/L to 5 g/1, 1.0 g/L to 250 g/L, and 20 g/L to 100 g/L.

Suitably the protein for use in the present invention is derived from natural sources.

Casein and whey protein are typically extracted from whole milk. Whey protein is a by-product of cheese manufacture. Ovalbumin is typically extracted from avian eggs, for example, chicken's eggs. Soy protein is typically extracted from soy beans.

An advantage presented by use according to the invention of acidic compositions containing protein is that the dental erosion potential of the compositions due to their acid content is reduced.

Suitably the pH of an acidic composition for use according to the present invention is in the range 2.0 to 4.5, more suitably 2.8 to 4.0. Most suitably the pH of an acidic composition for use according to the present invention is in the range 3.0 to 4.0.

Acidic compositions containing protein for use according to the invention may further comprise calcium in an amount from 0.01 mmol/L to 50 mmol/L, suitably from 2.0 mmol/L to 20 mmol/L.

An advantage presented by use according to the invention of acidic compositions containing protein and calcium, is that there is an enhanced reduction in the erosion potential of the acidic composition.

Calcium may be present in the form of a salt such as calcium carbonate, calcium hydroxide, calcium citrate, calcium malate, calcium citrate maleate, calcium lactate, calcium chloride, calcium glycerophosphate or calcium formate or any other salt.

An acidic composition containing protein for use according to the invention will typically contain an acidulant which is a dietary acid which may be an organic and/or inorganic acid. Typical dietary acids include for example, fruit acids such as citric and malic acid; potable acids such as lactic, phosphoric, acetic and tartaric acids and mixtures of one or more thereof.

Typically the concentration of acidulant in compositions containing protein for use according the invention, for example the citric acid or malic acid concentration in a fruit-based product, would be in the range 0.01% w/v to 4.0% w/v, suitably in the range 0.1% w/v to 1.0% w/v.

Acidic compositions containing protein for use according to the invention may be compositions intended for oral consumption in liquid, solid or semi-solid form such as acidic beverages, fruit juices, ciders, wines, vinegars and pickles boiled sweets, candies, tablets, lozenges, lollies, chews, jellies, gums, drops, dry powder blends such as powdered drinks intended for dissolution, eg. in water, and the like. Semi-solid products include dairy products such as yoghurts and set or frozen drinks.

Where the acidic composition containing protein according to the invention is a solid, the term pH means the pH of the composition before solidification, (where the composition is prepared via a liquid phase intermediate), or the pH of the composition when reconstituted or dissolved in a liquid, eg. water. The term solidification encompasses the treatment or supplementation of liquid phase intermediates to form a solid or semi-solid.

Acidic liquid compositions containing protein according to the invention for oral consumption may be in the form a ready to drink beverage, or a liquid concentrate for dilution with a liquid, such as for the preparation of a beverage that is ready to drink.

Acidic compositions containing protein according to the invention may also be in the form of a solid concentrate such as a dry powder for reconstitution with a liquid for the preparation of a beverage that is ready to drink.

Acidic compositions containing protein according to the invention may also be in the form of an oral care composition, such as a mouthwash, gel or spray.

Acidic oral care compositions containing protein for use according to the invention will contain appropriate formulating agents such as surfactants, thickening agents, humectants, flavouring agents, sweetening agents, opacifying or colouring agents, preservatives and water, selected from those conventionally used in the oral care composition art for such purposes. Examples of such agents are as described in EP 929287.

Oral care actives may also be included in the compositions of the present invention.

In another aspect of the present invention there is provided a method of reducing the dental erosion properties of an acidic oral composition having a pH in the range 2.0 to 5.0 which method comprises adding a protein selected from one or more of casein, ovalbumin, whey protein and soy protein to the acidic oral composition.

The acidic oral composition for use according to the invention can be prepared by admixing a protein selected from one or more of casein, ovalbumin, whey protein and soy protein with an acidulant and other ingredients and adjusting the pH in the range 2.0 to 5.0. In another aspect of the present invention there is provided a method of reducing dental erosion caused by exposure to an acidulant comprising orally administering a composition comprising an acidulant and a protein selected from one or more of casein, ovalbumin, whey protein and soy protein and having a pH in the range 2.0 to 5.0 to an individual in need thereof.

The present invention is illustrated by way of the following non-limiting examples.

### EXAMPLE 1

### Modification of an Acidic Composition to Reduce the Erosion Potential of a Composition

### Materials and Methods

### Hydroxyapatite Dissolution Measurements

An automatic titration system (Metrohm, Buckingham, UK), equipped with a 50 mL water-jacketed reaction vessel was set up to maintain constant pH by addition of 50 mmol/L HCl. The reaction temperature was 36° C. Hydroxyapatite was employed as an analogue of dental enamel (Barbour and Rees (2004) J. Dentistry 32: 591-602). Hydroxyapatite (HA) discs (Hitemco Medical Applications, Old Bethpage, USA), 12.05 mm in diameter×1.25 mm thick were fixed to a glass rod using sticky wax and the underside of the disc coated with nail varnish to give a constant exposed area of 155.5 mm² for each disc. The glass rod carrying the disc was fixed to a tube which fitted the inlet port of the reaction vessel lid and which held the specimen in a reproducible position with respect to the stirrer. For each dissolution measurement 15 mL of test solution was placed in the reaction vessel and stirred with a magnetic stirrer. When the temperature and pH had reached equilibrium the reaction was initiated by immersing the HA disc into the solution. Acid addition was linear with time and the rate of acid addition was used as the measure of HA dissolution.

New discs were conditioned by exposing them to stirred control citric acid solution for 30 min to remove any loosely attached or more soluble material. For every measurement, a control run and a test run were performed. The dissolution rate of an HA disc in the control citric acid solution was measured first and then the dissolution rate of the same HA disc in the test solution. Every measurement thus had its own control. Each test solution was tested three times. After a disc had been exposed to a protein test solution it was discarded.

### Citric Acid Solutions

All solutions for hydroxyapatite dissolution measurements were based on 0.3% w/v citric acid monohydrate solution, with pH adjusted to the appropriate value using KOH or HCl. Sodium caseinate (B D H, Poole, UK) was dissolved in distilled water by raising the pH using KOH until the casein dissolved. Stock 30% citric acid monohydrate solution was then added and the volume and the pH adjusted, giving a solution of 0.3% w/v citric acid monohydrate and the appropriate pH and concentration of casein. Ovalbumin solutions were prepared by dissolving the required amount of ovalbumin (M P Biochemicals, Ohio, USA) in distilled water using moderate stirring at room temperature for at least 60 min. Concentrated citric acid solution was then added to the protein solution to obtain a final concentration of 0.3% w/v citric acid monohydrate, and the pH was adjusted to the required value using KOH. Soy protein solutions were prepared by slowly adding weighed amounts to a stirred volume of about 80 mL of deionised water. The suspension was stirred until solubility equilibrium appeared to have been reached (about 30 min). 1 mL of 30% citric acid monohydrate was then added and the suspension stirred for a further 20 min. The suspension was centrifuged (4000 rpm, 15 min), the supernatant decanted off and made up to 100 mL with distilled water.

### The Effects of pH

Citric acid solutions as described above were prepared with no protein, and with either 0.2% w/v or 0.02% w/v protein, at pH 2.80, 3.00, 3.20, 3.40, 3.60 and 4.00.

### The Effects of Protein Concentration

Citric acid solutions as described above were prepared at pH 3.20 and protein concentrations of 0.02% w/v and 0.2% w/v.

### The Effects of Calcium

Citric acid solutions as described above were prepared at pH 3.20, and either no protein or a protein concentration of 0.2% w/v or 0.02% w/v. Calcium chloride dihydrate was added to achieve a calcium concentration of 5 mmol·L⁻¹, 10 mmol·L⁻¹, 20 mmol·L⁻¹ and 50 mmol·L⁻¹.

### Persistence of Action of Protein

To evaluate the persistence of the action of the protein against acid erosion, sequential measurements were performed. After conditioning the disc, three control runs of 30 minutes in the control citric acid solution were performed to obtain a mean baseline measurement. After this a single run of 30 minutes was performed in the acid solution with the protein. Following this, sequential 30 minute runs in the control acid solution were performed until the dissolution rate reached the baseline level. Each persistence of action experiment was performed at least three times with 0.2% w/v or 0.02% w/v protein each sequence being run on a separate HA disc.

### Data/Statistical Analysis

Hydroxyapatite dissolution rates were calculated from the rate of addition of H⁺ ions to the solution. Mean rates are expressed as nmol hydroxyapatite dissolved per min, per mm² of surface exposed. Standard deviations are represented by error bars.

### Results

### 1. Hydroxyapatite Dissolution

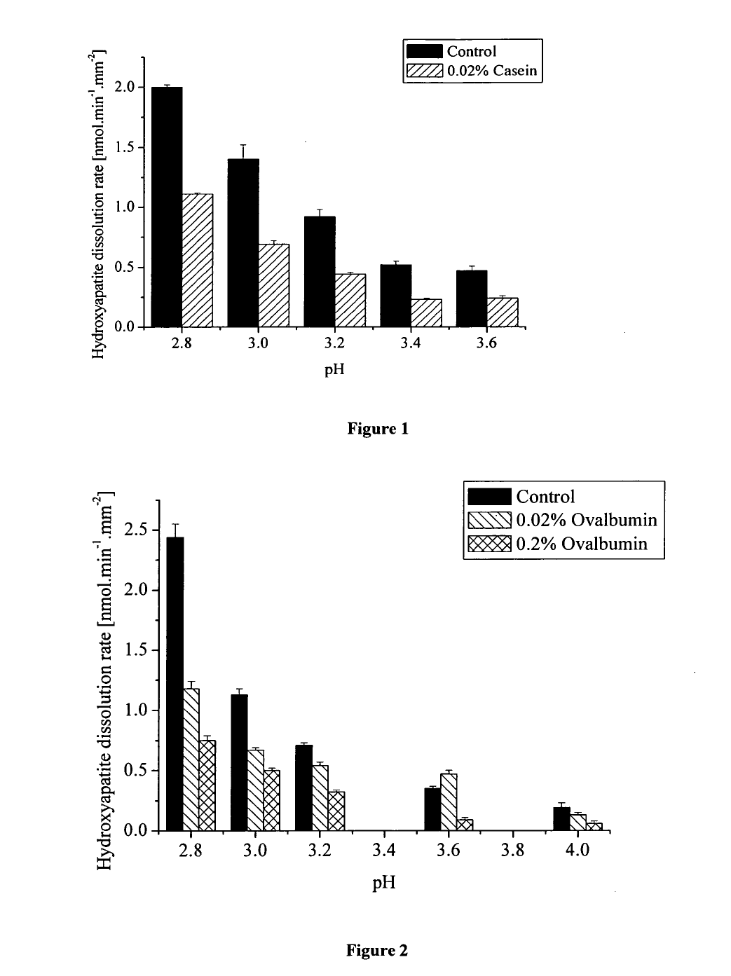

FIG. 1 shows the Hydroxyapatite dissolution rate in the presence of 0.02% w/v Casein. 0.3% w/v Citric acid, at pH 2.8, 3.0, 3.2, 3.40, 3.60.

TABLE 1
Hydroxyapatite dissolution rate in the presence of 0.02% w/v Casein. 0.3% w/v
citric acid, at pH 2.8, 3.0, 3.2, 3.40, 3.60. (Standard deviation in brackets)

| | pH | | | | |
|---|---|---|---|---|---|
| | 2.8 | 3.0 | 3.2 | 3.4 | 3.6 |
| No Casein | 2.00 ± 0.04 | 1.40 ± 0.20 | 0.92 ± 0.11 | 0.52 ± 0.06 | 0.47 ± 0.06 |
| 0.02% Casein | 1.11 ± 0.02 | 0.69 ± 0.05 | 0.44 ± 0.04 | 0.23 ± 0.01 | 0.24 ± 0.04 |

FIG. 2 shows the Hydroxyapatite dissolution rate in the presence of 0.02% w/v and 0.2% w/v Ovalbumin. 0.3% w/v citric acid. pH 2.8, 3.0, 3.2, 3.60, 4.0.

TABLE 2
Hydroxyapatite dissolution rate in the presence of 0.02% w/v and 0.2% w/v
Ovalbumin. 0.3% w/v citric acid. pH 2.8, 3.0, 3.2, 3.60, 4.0.
(Standard deviation in brackets).

| | pH | | | | |
|---|---|---|---|---|---|
| | 2.8 | 3.0 | 3.2 | 3.6 | 4.0 |
| No Ovalbumin | 2.44 ± 0.21 | 1.13 ± 0.09 | 0.71 ± 0.04 | 0.35 ± 0.03 | 0.19 ± 0.07 |
| 0.02% | 1.18 ± 0.11 | 0.67 ± 0.03 | 0.54 ± 0.06 | 0.47 ± 0.05 | 0.13 ± 0.01 |
| Ovalbumin | | | | | |
| 0.2% Ovalbumin | 0.75 ± 0.08 | 0.50 ± 0.02 | 0.32 ± 0.04 | 0.09 ± 0.01 | 0.06 ± 0.01 |

### 2. Effect of Calcium Concentration

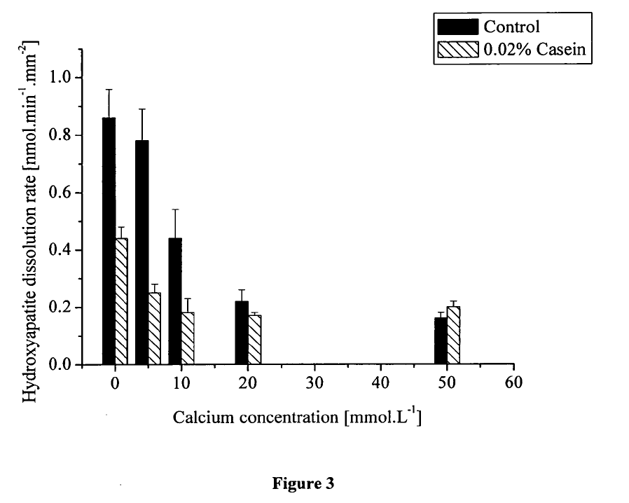

FIG. 3 shows the effect of calcium concentration on Hydroxyapatite dissolution in the presence of 0.02% w/v Casein. 0.3% w/v Citric acid monohydrate, pH 3.20.

TABLE 3
Effect of calcium concentration on Hydroxyapatite dissolution in the presence of
0.02% w/v Casein. 0.3% w/v Citric acid monohydrate, pH 3.20.
(Standard deviation in brackets).

| | Calcium [mmol/L] | | | | |
|---|---|---|---|---|---|
| | 0 | 5 | 10 | 20 | 50 |
| No Casein | 0.86 ± 0.11 | 0.78 ± 0.12 | 0.44 ± 0.10 | 0.22 ± 0.04 | 0.16 ± 0.02 |
| 0.02% Casein | 0.44 ± 0.04 | 0.25 ± 0.03 | 0.18 ± 0.05 | 0.17 ± 0.01 | 0.20 ± 0.02 |

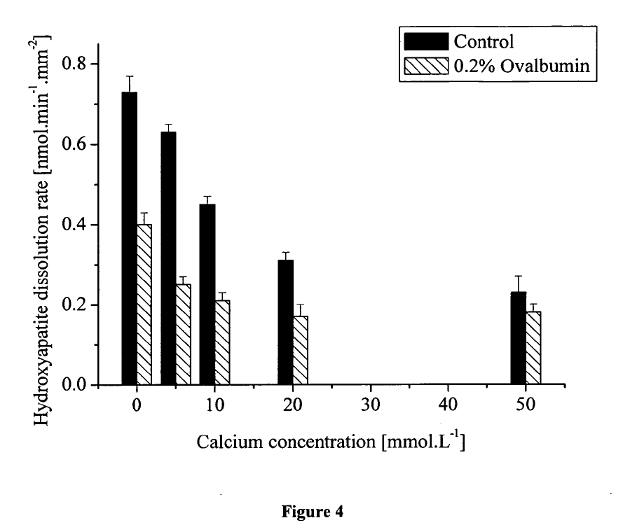

FIG. 4 shows the effect of calcium concentration on Hydroxyapatite dissolution in the presence of 0.2% w/v Ovalbumin. 0.3% w/v Citric acid monohydrate, pH 3.2.

TABLE 4
Effect of calcium concentration on Hydroxyapatite dissolution in the presence of
0.2% w/v Ovalbumin. 0.3% w/v Citric acid monohydrate, pH 3.2.
(Standard deviation in brackets).

| | Calcium [mmol/L] | | | | |
|---|---|---|---|---|---|
| | 0 | 5 | 10 | 20 | 50 |
| No Ovalbumin | 0.73 ± 0.06 | 0.63 ± 0.03 | 0.45 ± 0.04 | 0.31 ± 0.03 | 0.23 ± 0.06 |
| 0.2% Ovalbumin | 0.40 ± 0.05 | 0.25 ± 0.03 | 0.21 ± 0.03 | 0.17 ± 0.04 | 0.18 ± 0.02 |

### 3. Persistence of Effect

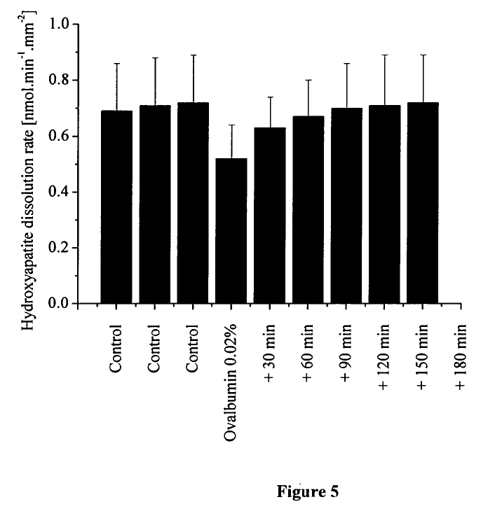

FIG. 5 shows the effect of 0.02% w/v Ovalbumin on Hydroxyapatite dissolution 0.3% w/v Citric acid over 150 mins.

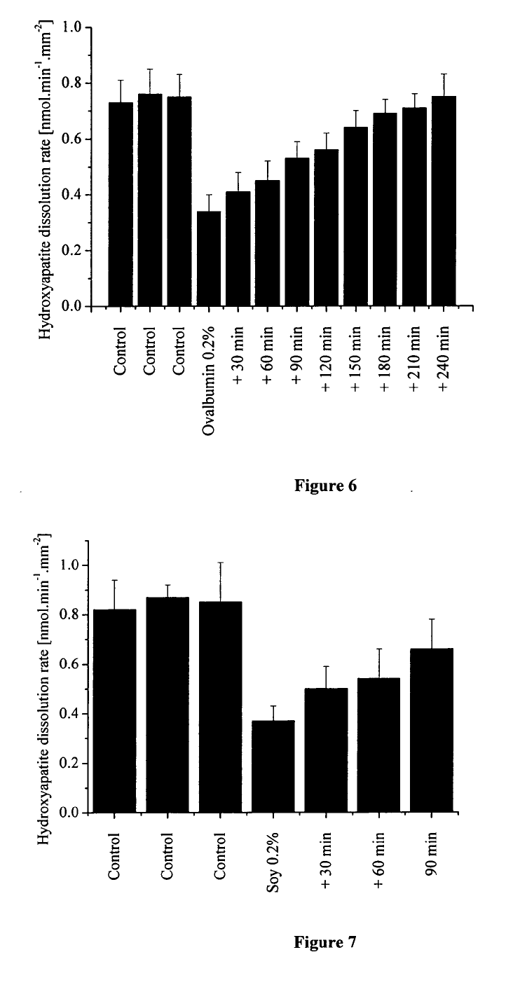

FIG. 6 shows the effect of 0.2% w/v Ovalbumin on Hydroxyapatite dissolution 0.3% w/v Citric acid over 240 mins.

TABLE 5
Effect of 0.02% & 0.2% w/v Ovalbumin (OA) on Hydroxyapatite dissolution
0.3% w/v Citric acid, pH 3.2 over 150 mins and 240 mins. (Standard deviation in brackets).

| | | | | | | | +90 | +120 | +150 | +180 | +210 | +240 |
|---|---|---|---|---|---|---|---|---|---|---|---|---|
| | control | control | control | OA | +30 min | +60 min | min | min | min | min | min | min |
| 0.02% | 0.69 ± 0.17 | 0.71 ± 0.17 | 0.72 ± 0.17 | 0.52 ± 0.12 | 0.63 ± | 0.67 ± | 0.70 ± | 0.71 ± 0.18 | 0.72 ± | | | |
| OA | | | | | 0.11 | 0.13 | 0.16 | | 0.16 | | | |
| 0.2% | 0.73 ± 0.15 | 0.76 ± 0.17 | 0.75 ± 0.15 | 0.34 ± 0.12 | 0.41 ± | 0.45 ± | 0.53 ± | 0.56 ± 0.12 | 0.64 ± | 0.69 ± | 0.71 ± | 0.75 ± |
| OA | | | | | 0.14 | 0.13 | 0.11 | | 0.12 | 0.10 | 0.10 | 0.16 |

FIG. 7 shows the effect of 0.2% w/v Soy protein on Hydroxyapatite dissolution 0.3% w/v Citric acid, pH 3.2 over 90 min

TABLE 6
Effect of 0.2% w/v Soy protein on Hydroxyapatite dissolution 0.3% w/v Citric
acid, pH 3.2 over 90 min. (Standard deviation in brackets).

| | control | control | control | 0.2% Soy | 30 min | 60 min | 90 min |
|---|---|---|---|---|---|---|---|
| 0.2% Soy | 0.82 ± 0.12 | 0.87 ± 0.05 | 0.85 ± ±0.16 | 0.37 ± 0.06 | 0.50 ± 0.09 | 0.54 ± 0.12 | 0.66 ± 0.12 |
| protein | | | | | | | |

### SUMMARY

Example 1 clearly demonstrates that an acidic solution modified with the addition of protein has a reduced erosion potential and is thus less erosive to teeth.

### EXAMPLE 2a

### Typical Beverage Formulation—0.02% w/v Protein

| Ingredient | Amount g/l | Amount % w/v |
|---|---|---|
| Protein source | 0.200 | 0.020 |
| Carbohydrate blend | 102.920 | 10.292 |
| Liquid sugar | 72.620 | 7.262 |
| Fruit Juice | 12.070 | 1.207 |
| Citric acid anhydrous | 0.750 | 0.075 |
| Ascorbic acid | 0.570 | 0.057 |
| Colour | 0.54 | 0.054 |
| Water | To 1 L | 81.033 |

pH 3.2, acidity 0.3% w/v CAMH

### EXAMPLE 2b

### Typical Beverage Formulation—0.02% w/v Protein

| Ingredient | Amount g/l | Amount % w/v |
|---|---|---|
| Protein source | 0.200 | 0.020 |
| Carbohydrate blend | 102.920 | 10.292 |
| Liquid sugar | 72.620 | 7.262 |
| Fruit Juice | 12.070 | 1.207 |
| Citric acid anhydrous | 2.8 | 0.28 |
| Flavouring | 0.2 | 0.02 |
| Colour | 0.9 | 0.09 |
| Water | To 1 L | 80.829 |

### EXAMPLE 3a

### Typical Beverage Formulation—2.0% w/v Protein

| Ingredient | Amount g/l | Amount % w/v |
|---|---|---|
| Protein source | 20.00 | 0.020 |
| Aspartame | 0.320 | 0.032 |
| Acesulfame K | 0.110 | 0.011 |
| Fruit Juice | 12.070 | 1.207 |
| Citric acid anhydrous | 0.750 | 0.075 |
| Ascorbic acid | 0.570 | 0.057 |
| Flavour | 0.220 | 0.022 |
| Colour | 0.540 | 0.054 |
| Water | To 1 L | 98.522 |

pH 3.2, acidity 0.3% w/v CAMH

### EXAMPLE 3b

### Typical Beverage Formulation—2.0% w/v Protein

| Ingredient | Amount g/l | Amount % w/v |
|---|---|---|
| Protein source | 20.00 | 2.000 |
| Aspartame | 0.320 | 0.032 |
| Acesulfame K | 0.110 | 0.011 |
| Fruit Juice | 12.070 | 1.207 |
| Citric acid anhydrous | 2.8 | 0.28 |
| Flavour | 0.200 | 0.020 |
| Colour | 0.9 | 0.09 |
| Water | To 1 L | 96.36 |

1.-5. (canceled)

6. An orally administrable composition comprising an acidulant and a protein selected from one or more of casein, ovalbumin, whey protein and soy protein and having a pH in the range 2.0 to 5.0 for use in the reduction of dental erosion caused by exposure to the acidulant.

7. An orally administrable composition as claimed in claim 6 wherein the pH is in the range 2.0 to 4.5.

8. An orally administrable composition according to claim 6 wherein protein is present in an amount from 0.01% w/v to 50% w/v.

9. An orally administrable composition according to claim 6 wherein the protein is present in an amount from 0.10 g/L to 500 g/L.

10. An orally administrable composition according to claim 6 further comprising calcium in an amount from 0.01 mmol/L to 50 mmol/L.

11. A method of reducing the dental erosion properties of an acidic oral composition having a pH in the range 2.0 to 5.0 which method comprises adding a protein selected from one or more of case in, ovalbumin, whey protein and soy protein to the acidic oral composition.

12. A method of reducing dental erosion caused by exposure to an acidulant comprising orally administering a composition comprising an acidulant and a protein selected from one or more of casein, ovalbumin, whey protein and soy protein and having a pH in the range 2.0 to 5.0 to an individual in need thereof.
